(19) United States
(12) Patent Application Publication
(10) Pub. No.: US 2014/0081341 A1
(43) Pub. Date: Mar. 20, 2014

(54) BONE FIXATION GUIDE DEVICE

(71) Applicants: CHUN-LI LIN, TAOYUAN COUNTY (TW); ALVIN CHAO-YU CHEN, TAOYUAN COUNTY (TW); METAL INDUSTRIES RESEARCH&DEVELOPMENT CENTRE, KAOHSIUNG (TW)

(72) Inventors: CHUN-LI LIN, TAOYUAN COUNTY (TW); YU-HAO LIN, KAOHSIUNG CITY (TW); ALVIN CHAO-YU CHEN, TAOYUAN COUNTY (TW); TSUNG-CHIH YU, TAINAN CITY (TW); SHIEN-NAN KUO, KAOHSIUNG CITY (TW)

(73) Assignees: CHUN-LI LIN, TAOYUAN COUNTY (TW); METAL INDUSTRIES RESEARCH&DEVELOPMENT CENTRE, KAOHSIUNG (TW); ALVIN CHAO-YU CHEN, TAOYUAN COUNTY (TW)

(21) Appl. No.: 14/027,499

(22) Filed: Sep. 16, 2013

(30) Foreign Application Priority Data
Sep. 17, 2012 (TW) 101134014

(51) Int. Cl. A61B 17/80

(52) U.S. Cl. CPC A61B 17/808
USPC 606/86 B

(57) ABSTRACT

A bone fixation guide device is adapted for fixing a bone plate on a bone, wherein the bone plate has at least two screw holes. The bone fixation guide device includes: a handheld holder having a through hole; a first guide sleeve having a guide hole, adapted for passing through the through hole of the handheld holder, and fixed to the bone plate; and a guide plate having a fixing portion and at least two openings, the fixing portion adapted for being assembled on the first guide sleeve, whereby the guide plate is disposed on the handheld holder, wherein the screw holes of the bone plate correspond to the openings of the guide plate.

### CROSS-REFERENCE TO RELATED APPLICATION

This application claims the benefit of Taiwan Patent Application No. 101134014, filed on Sep. 17, 2012, which is hereby incorporated by reference for all purposes as if fully set forth herein.

### BACKGROUND OF THE INVENTION

1. Field of the Invention

The present invention relates to a bone fixation guide device, and in particular, to a bone fixation guide device for fixing a bone plate on a bone.

2. Related Art

In recent years, a minimally invasive surgery has gradually replaced large-incision surgery. The minimally invasive surgery, as the name implies, achieves a same treatment effect with a smaller incision, and also tends to be applied to surgeries such as fracture reduction and joint replacement in orthopedic treatments. Therefore, compared with a large incision, the minimally invasive surgery has an advantage that: there is a smaller incision and thus a smaller scar after recovery, also less soft tissue damage occurs and fewer scabs are formed resulting in less pain and a better recovery effect, and fewer complications might occur.

Generally, the minimally invasive surgery requires repeated irradiation and positioning with an X-ray, which takes a long time, and many times of X-ray irradiation easily causes abnormal growth of cells.

U.S. Pat. No. 7,153,309 discloses a guide system for bone-repair devices, which can be used to guide a drill bit and bone screws for drilling and locking in a pre-defined direction relative to a bone plate. For patients with osteoporosis, the bone screws can provide sufficient grip for the bone to pull fractured bones in the direction of the bone plate, so as to fix the positions of the bone and the bone plate. However, the surgery disclosed on this patent requires incisions on both opposite surfaces of the bone, such that it cannot achieve the purpose of treatment of the minimally invasive surgery.

U.S. Pat. No. 7,648,508 discloses bone plating implants, instruments and methods, and devices those can accurately implant bone screws and bone plates at necessary positions. However, when the bone plating implants, instruments and methods disclosed on this patent are used, a surgeon has to add a space for placing a handle during a medical operation, so as to increase the area of a surgical incision.

Therefore, it is necessary to provide a bone fixation guide device, so as to solve the above problem.

### SUMMARY OF THE INVENTION

An objective of the present invention is to provide a bone fixation guide device, so as to accurately implant bone screws and bone plates at necessary positions, and avoid a large incision and a long operation time.

To achieve the above objectives, the present invention provides a bone fixation guide device adapted for fixing a bone plate on a bone, wherein the bone plate has at least two screw holes. The bone fixation guide device includes: a handheld holder having a through hole; a first guide sleeve having a guide hole, adapted for passing through the through hole of the handheld holder, and fixed to the bone plate; and a guide plate having a fixing portion and at least two openings, the fixing portion adapted for being assembled on the first guide sleeve, whereby the guide plate is disposed on the handheld holder, wherein the screw holes of the bone plate correspond to the openings of the guide plate.

The bone fixation guide device of the present invention can accurately position the screw holes of the bone plate under the skin, reduce the size of a surgical incision, and shorten an operation time, so as to effectively help a clinician to perform an endosseous fixation surgery, reduce complications, improve the operation efficiency, and decrease the waste of medical resources.

The bone fixation guide device of the present invention can be used to perform a minimally invasive surgery for fracture fixation, and can place the bone plate at a subcutaneous focus by combining the handheld holder, the first guide sleeve, and the bone plate. After the handheld portion, the first guide sleeve, and the bone plate are combined, the positions of openings of the guide plate correspond to the positions of screw holes of the bone plate under the skin. The guide probe is then used to determine an approximate position of a following bone screw on the skin, and the second guide sleeve first passes through the skin and then is combined with the bone plate, so as to perform drilling and to accurately lock the bone screws into a bone body vertically. Therefore, the design of the bone fixation guide device has the following advantages: 1. The bone fixation guide device has a simple structure, is easy to be operated, and can improve the operation efficiency. 2. The bone fixation guide device can be used with surgical instruments, so as to accurately obtain the screw holes of the bone plate under the skin and perform drilling immediately without having to remove the bone fixation guide device after surgical alignment, and thus to improve surgical convenience. 3. The size of the incision is reduced, and the time that a patient is exposed to an X-ray and the operation time are shortened.

To make the above and other objectives, features, and advantages of the present invention more comprehensible, the present invention is described below in detail with reference to the accompanying drawings.

### BRIEF DESCRIPTION OF THE DRAWINGS

FIG. 1a is a schematic perspective view of a handheld holder of a bone fixation guide device according to the present invention;

FIG. 1b is a schematic bottom view of the body of the handheld holder of the bone fixation guide device according to the present invention;

FIG. 1c is a schematic side view of a first guide sleeve of the bone fixation guide device according to the present invention;

FIG. 1d is a schematic perspective view of a guide plate of the bone fixation guide device according to the present invention;

FIG. 1e is a schematic side view of a first guide probe and a second guide probe of the bone fixation guide device according to the present invention;

FIG. 2a is a schematic perspective view of a sliding sleeve of the bone fixation guide device according to the present invention;

FIG. 2b is a schematic view of a sliding rod of the bone fixation guide device according to the present invention;

FIG. 2c is a schematic view of a second guide sleeve of the bone fixation guide device according to the present invention;

FIG. 3 is a schematic perspective view of a bone plate according to the present invention; and

FIG. 4a to FIG. 4n are schematic views showing manners of using the bone fixation guide device for fracture at the end of radius according to the present invention.

### DETAILED DESCRIPTION OF THE EMBODIMENTS

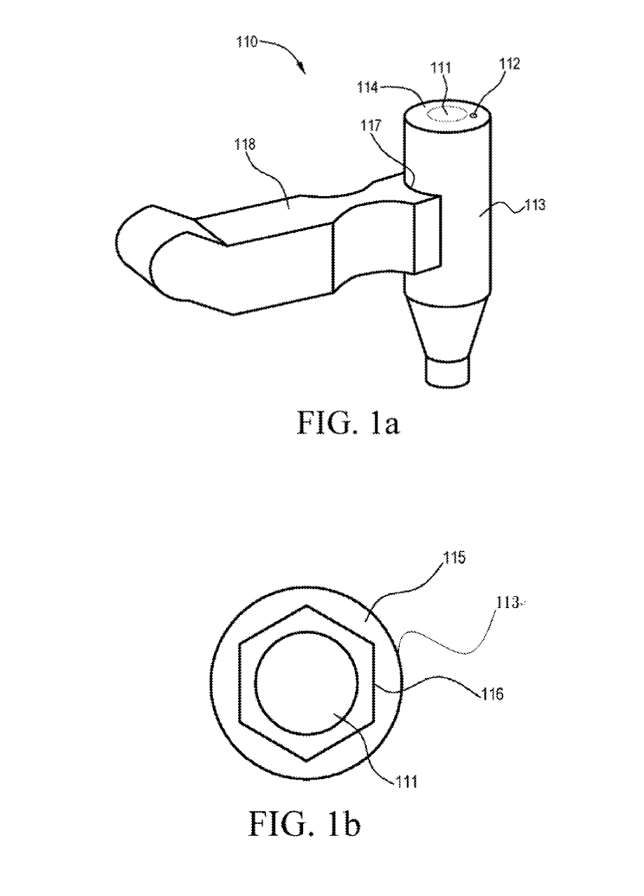

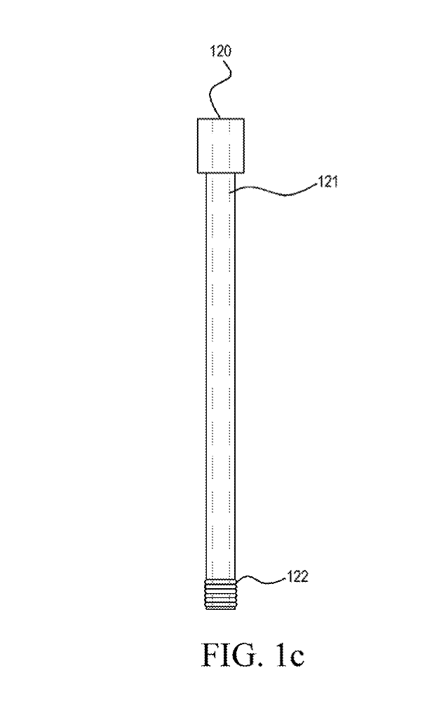

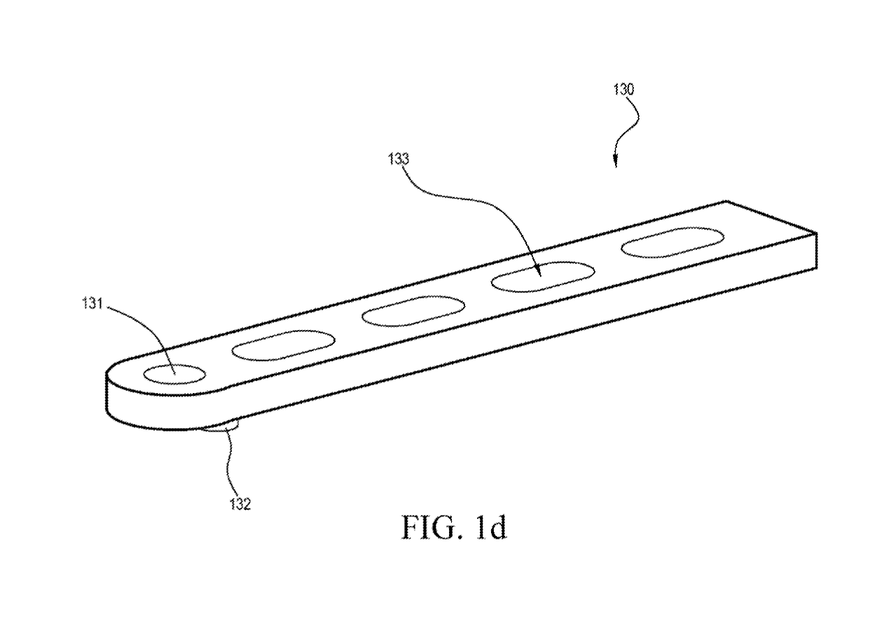

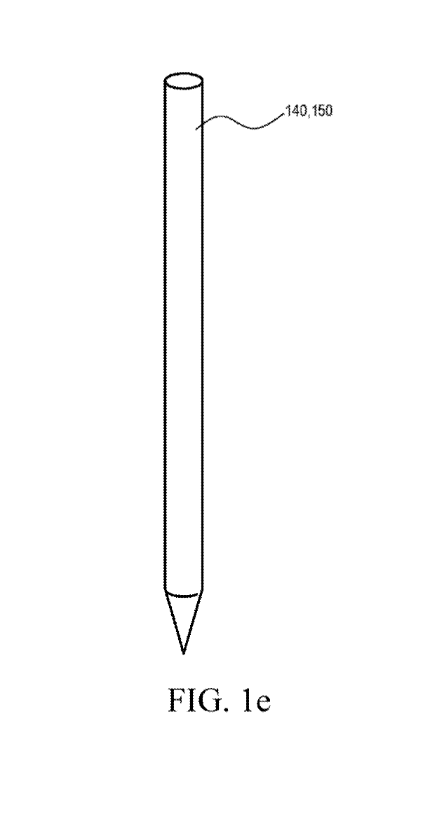

FIGS. 1a to 1f are schematic exploded views of a bone fixation guide device according to the present invention. The bone fixation guide device includes: a handheld holder 110 (shown in FIG. 1a), a first guide sleeve 120 (shown in FIG. 1c), and a guide plate 130 (shown in FIG. 1d).

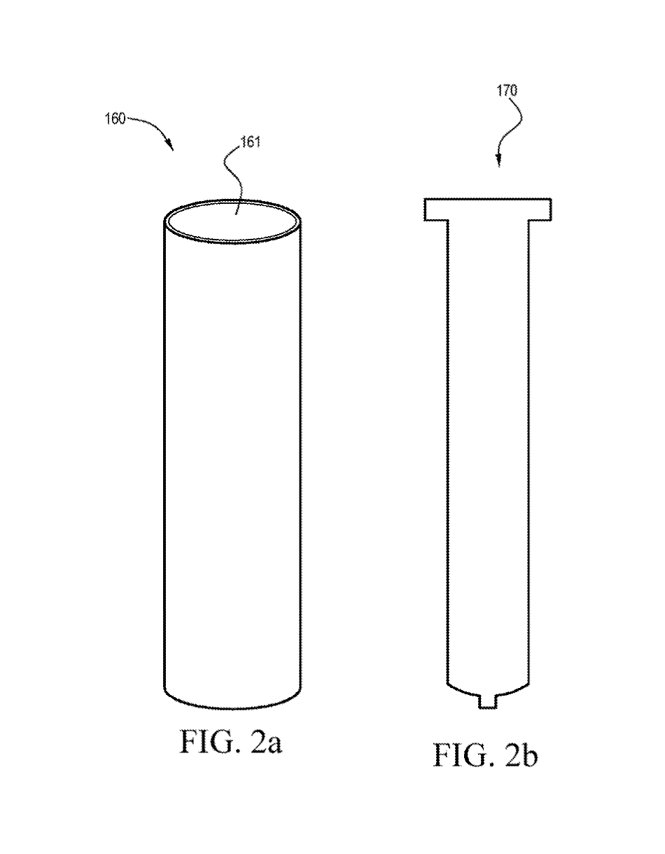

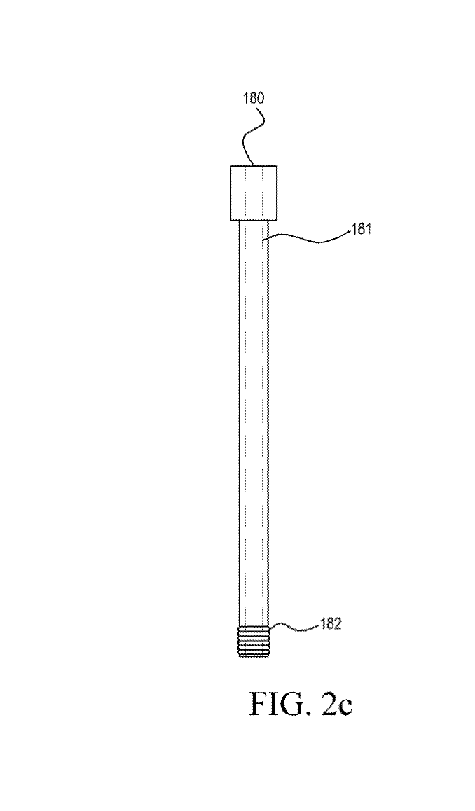

The bone fixation guide device in the present invention can further include a first guide probe 140 (shown in FIG. 1e), a marker, and a second guide sleeve 180 (shown in FIG. 2c).

The bone fixation guide device in the present invention can further include a sliding sleeve 160 (shown in FIG. 2a) and a sliding rod 170 (shown in FIG. 2b).

A through hole 111 is provided in a body 113 of the handheld holder 110. A positioning hole 112 is provided in an upper surface 114 of the body 113, and a positioning recess 116 (shown in FIG. 1b, for example, a hexagonal hollow recess, which may be a positioning protrusion, but the positioning recess is used to describe embodiments of the present invention) is provided in a lower surface 115 of the body 113. Also, a grip 118 is provided at a side surface 117 of the body 113 so as to be easily operated and forced by a user. The first guide sleeve 120 has a guide hole 121 and an external thread 122, and the second guide sleeve 180 has a guide hole 181 and an external thread 182. The guide plate 130 has a fixing portion 131, a positioning column 132, and a plurality of openings 133 (at least two openings 133). The sliding sleeve 160 has a through hole 161.

The marker is adapted to provide a mark on the skin, wherein the marker may be a pen or other objects that can make a mark, and in this embodiment, the marker is a second guide probe 180 (shown in FIG. 1e).

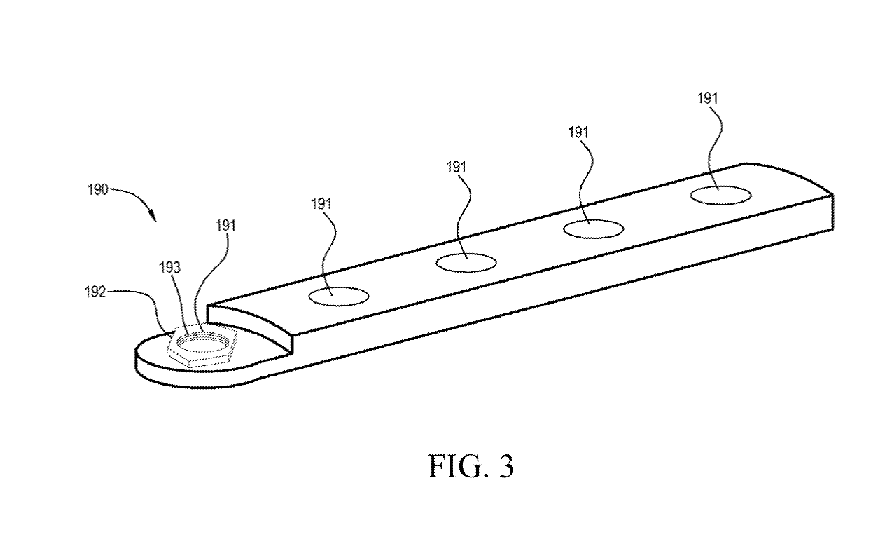

FIG. 3 is a schematic perspective view of a bone plate, and shows a bone plate used in the bone fixation guide device according to the present invention. The bone plate 190 includes a plurality of screw holes 191 (at least two screw holes 191) and a positioning protrusion 192 (for example, a hexagonal hollow protrusion, but the bone plate 190 has a positioning recess when a positioning protrusion is provided at the lower surface 115 of the handheld holder). The screw holes 191 are evenly distributed on the bone plate 190, and the positioning protrusion 192 is located at one side of the bone plate 190.

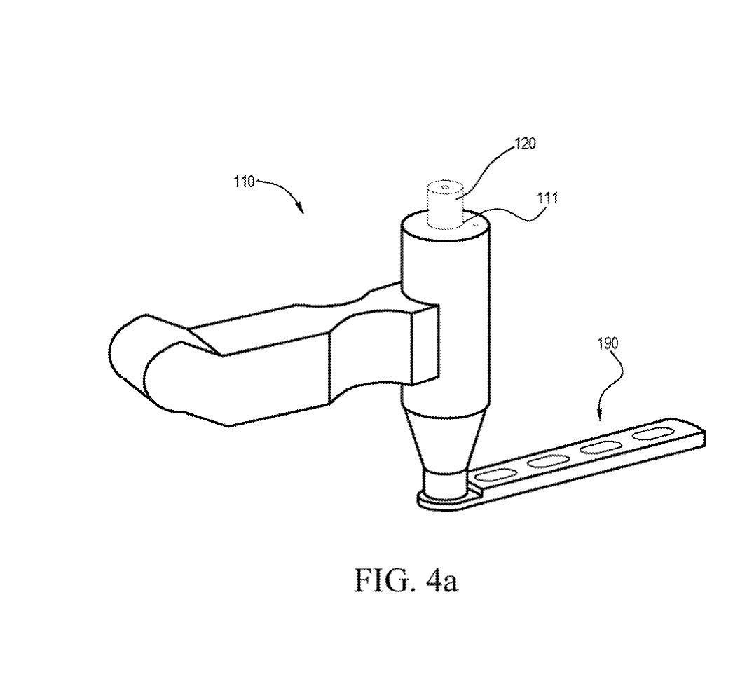

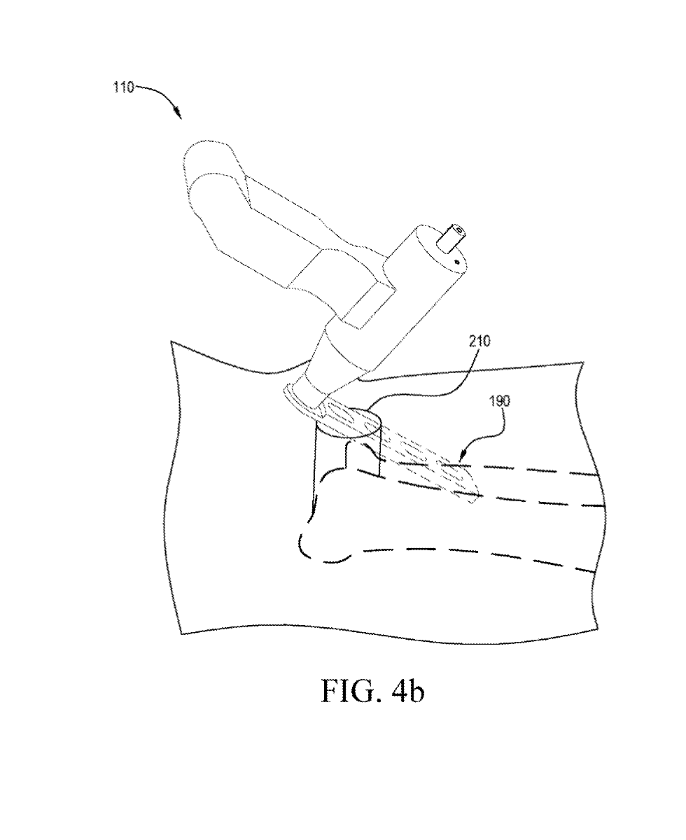

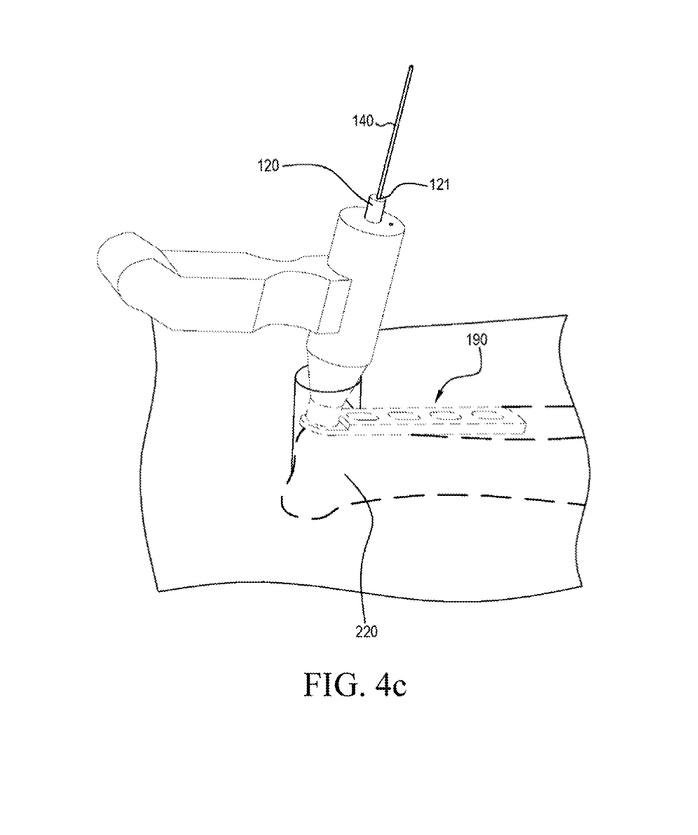

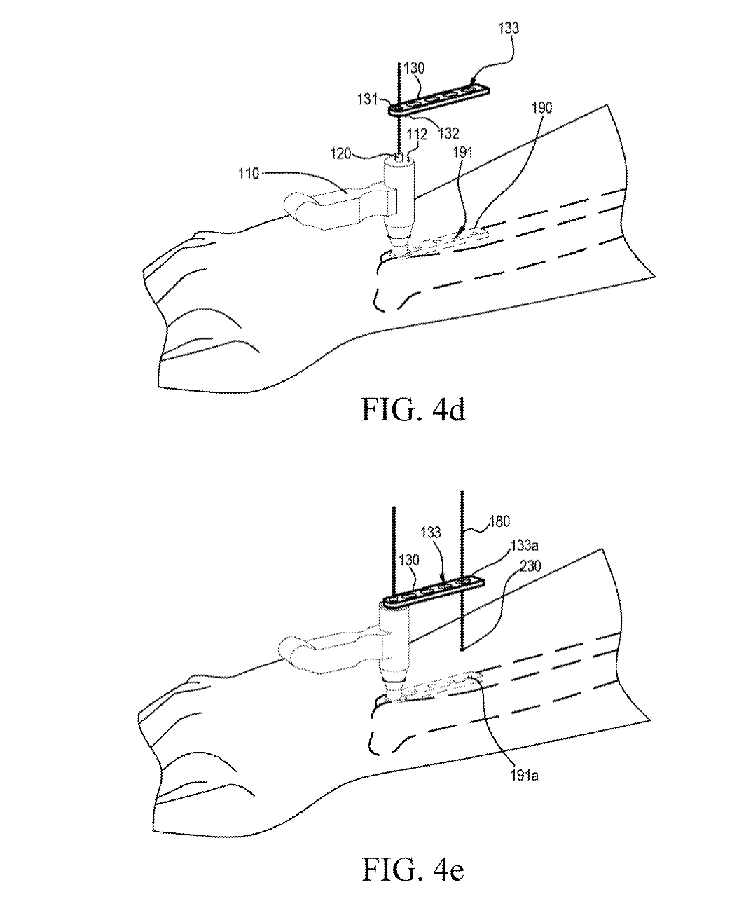

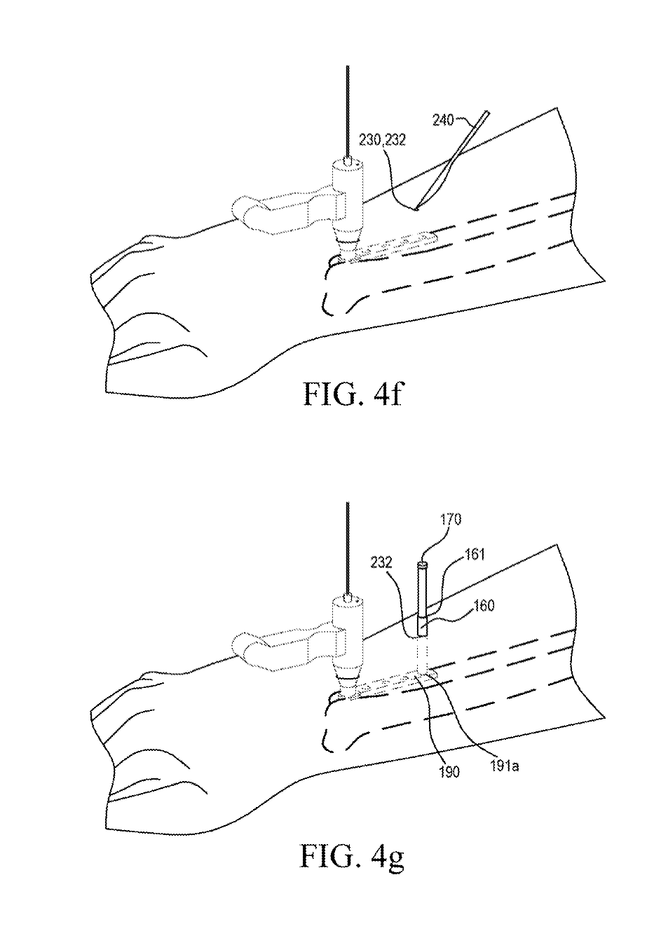

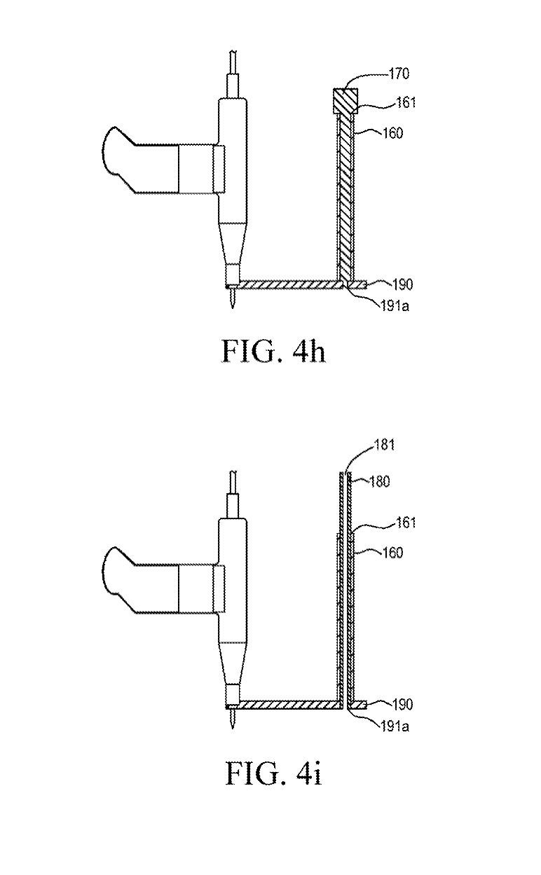

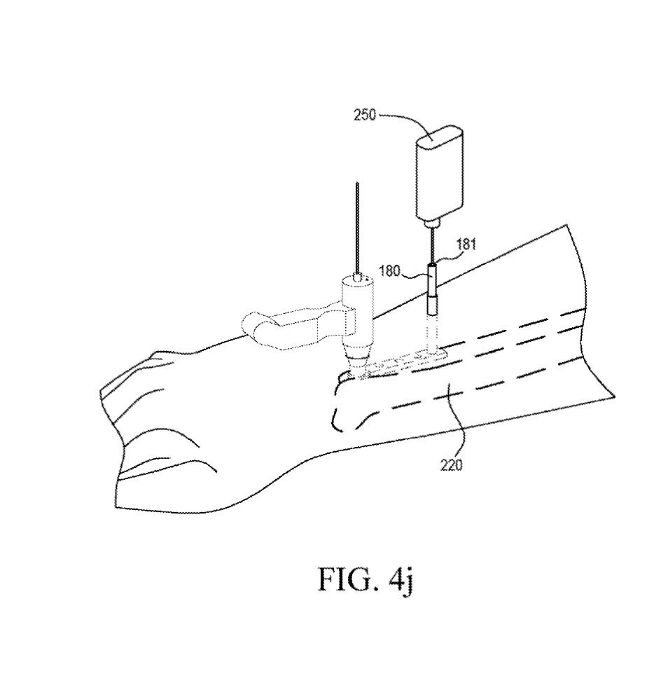

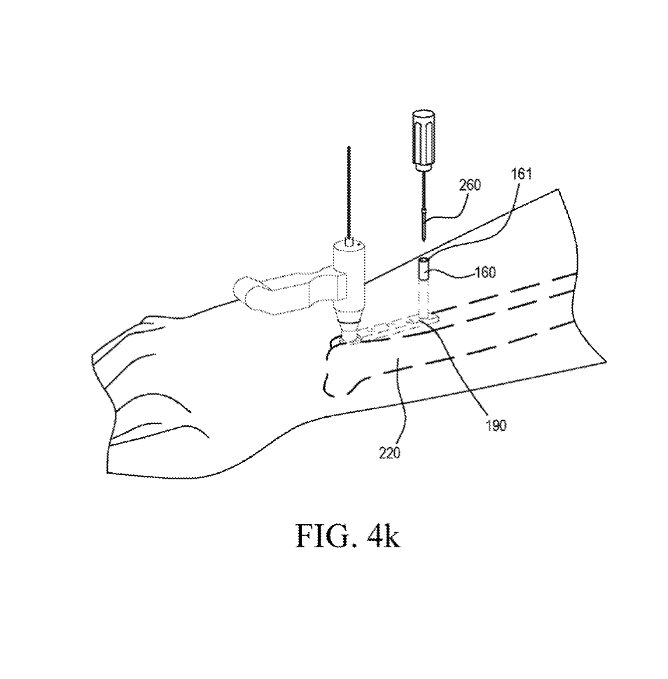

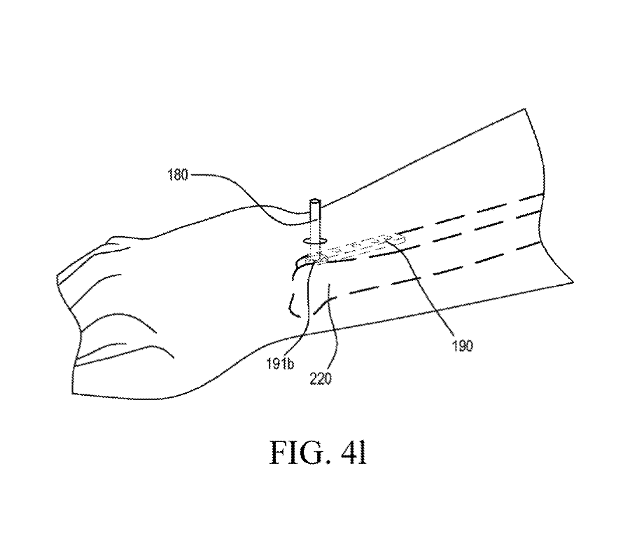

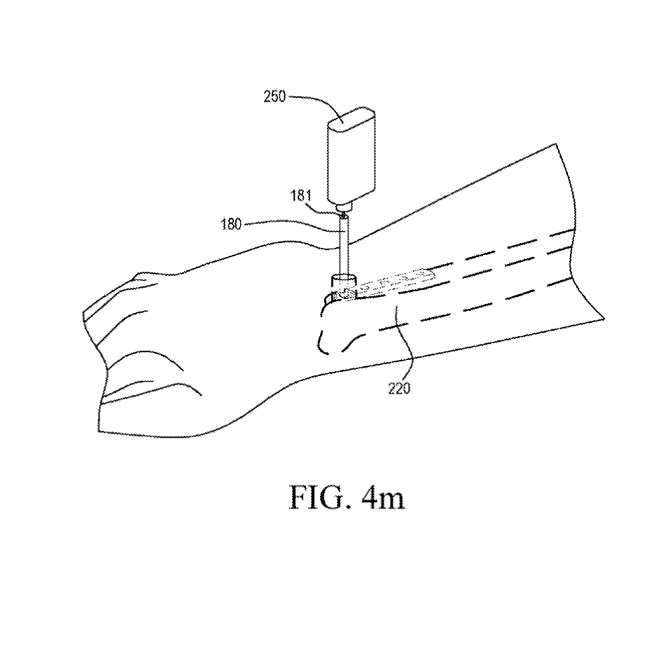

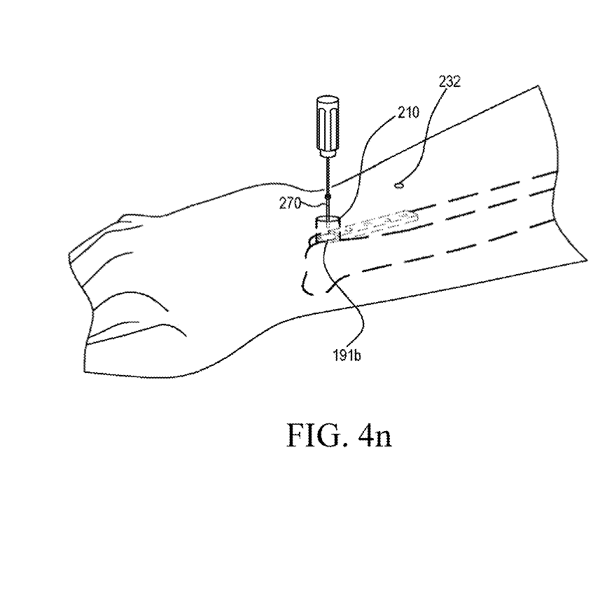

By taking fracture at the end of radius of an arm as an example, FIG. 4a to FIG. 4n are schematic views showing manners of using the device according to the present invention. According to the steps shown in FIG. 4a, one end where the first guide sleeve 120 has an external thread 122 (shown in FIG. 1c) passes through the through hole 111 of the handheld holder 110. When the first guide sleeve 120 contacts with the bone plate 190, the positioning protrusion 192 (shown in FIG. 1b) of the bone plate 190 is stuck in the positioning recess 116 (shown in FIG. 3) of the handheld holder 110 at first, and then the first guide sleeve 120 is fastened and fixed to the bone plate 190 by screwing the external thread 122 of the first guide sleeve 120 to an internal thread 193 of the positioning protrusion 192 of the bone plate 190.

According to the steps shown in FIG. 4b, a first incision 210 is first cut above the bone of the radius in this embodiment, and the bone plate 190 is placed in the arm through the first incision 210 by handling the handheld holder 110 and reaches a predetermined position.

According to the steps shown in FIG. 4c, a first guide probe 140 passes through the guide hole 121 of the first guide sleeve 120, and is drilled into the bone 220, so as to temporarily fix the position of the bone plate 190 on the bone 220.

According to the steps shown in FIG. 4d, when the fixing portion 131 of the guide plate 130 is assembled on the first guide sleeve 120, the positioning column 132 of the guide plate 130 is stuck in the positioning hole 112 of the handheld holder 110, so that the guide plate 130 is disposed on the handheld holder 110. Therefore, the guide plate 130 is located right above the bone plate 190, and the guide plate 130 and the bone plate 190 are parallel to each other. In detail, in this embodiment, the positioning protrusion 192 (shown in FIG. 3) of the bone plate 190 is stuck in the positioning recess 116 (shown in FIG. lb) of the handheld holder 110, and the positioning column 132 of the guide plate 130 is stuck in the positioning hole 112 of the handheld holder 110, so as to ensure that the guide plate 130 and the bone plate 190 are parallel to each other, so that the screw holes 191 of the bone plate 190 correspond to the openings 133 of the guide plate 130. In another embodiment, the bone plate 190 may include a positioning recess, the handheld holder 110 may include a positioning protrusion, the guide plate 130 may include a positioning hole, and the handheld holder 110 may include a positioning column, thereby may also achieving the effect of making the guide plate 130 and the bone plate 190 parallel to each other, so that the screw holes 191 of the bone plate 190 also correspond to the openings 133 of the guide plate 130.

According to the steps shown in FIG. 4e, the second guide probe 180 (i.e., the marker) passes through one opening 133a of the openings 133 of the guide plate 130, and pricks the skin to form a small hole 230 acted as a mark. Basically, the small hole 230 can be located above a screw hole 191a corresponding to the opening 133a. Therefore, the opening 133a of the guide plate 130 can be used for determining an approximate position of a following bone screw, and the fixing portion 131 of the guide plate 130 determines an approximate position of another following bone screw.

According to the steps shown in FIG. 4f, the second guide probe 180 and the guide plate 130 are removed, and a scalpel 240 is used to cut a second incision 232 at the small hole 230.

According to the steps shown in FIG. 4g, the sliding sleeve 160 first passes through the second incision 232 and contacts with the bone plate 190, so that the through hole 161 of the sliding sleeve 160 is located above the screw hole 191a of the bone plate 190. Afterwards, the sliding rod 170 passes through the through hole 161 of the sliding sleeve 160, and is inserted into the screw hole 191a of the bone plate 190, so as to confirm whether the through hole 161 of the sliding sleeve 160 is located right above the screw hole 191a.

According to the steps shown in FIG. 4h, after it is confirmed that the through hole 161 of the sliding sleeve 160 is located right above the screw hole 191a, the sliding rod 170 is removed. At this time, whether tissue cells remain in the screw hole 191a can be observed by the through hole 161 of the sliding sleeve 160.

According to the steps shown in FIG. 4i, the second guide sleeve 180 passes through the through hole 161 of the sliding sleeve 160, so that the guide hole 181 of the second guide sleeve 180 is located above the screw hole 191a of the bone plate 190, and is screwed and fixed to the screw hole 191a of the bone plate 190.

According to the steps shown in FIG. 4j, a drill 250 is used, and the drill 250 passes through the guide hole 181 of the second guide sleeve 180 and performs drilling of a proximal fixing hole in the bone 220, wherein the proximal fixing hole are close to the body and are an opening drilled in the bone 220 by the drill 250.

According to the steps shown in FIG. 4k, after the drill and the second guide sleeve are removed, the bone screw 260 passes through the through hole 161 of the sliding sleeve 160 and is locked in the proximal fixing hole of the bone 220, so as to fix the bone plate 190 on the bone 220.

According to the steps shown in FIG. 41, after the locking of the proximal fixing hole is completed, the handheld holder, the first guide sleeve, and the first guide probe are removed, and the second guide sleeve 180 is locked in a screw hole 191b of the bone plate 190, wherein the screw hole 191b is a screw hole away from the body.

According to the steps shown in FIG. 4m, the drill 250 is used to pass through the guide hole 181 of the second guide sleeve 180 and perform drilling on the bone 220, so as to drill a distal fixing hole on the bone 220, and the distal fixing hole are away from the body and are an opening drilled in the bone 220 by the drill 250.

According to the steps shown in FIG. 4n, after the second guide sleeve 180 is removed, the bone screw 270 is locked in the distal fixing hole. Finally, the first incision 210 and the second incision 232 are stitched up, and minimally invasive surgery of fracture at the end of radius is completed.

In another embodiment, the sliding sleeve 160 and the sliding rod 170 are not used. Therefore, the steps shown in FIG. 4g to FIG. 4i can be simplified as: the second guide sleeve 180 directly passes through the second incision 232, and is screwed in the screw hole 191a of the bone plate 190. Also, the step shown in FIG. 4k can be simplified as: after the drill and the second guide sleeve is removed, the bone screw 260 passes through the second incision 232 and is locked in the bone 220, so as to fix the bone plate 190 on the bone 220.

The bone fixation guide device of the present invention can accurately position the screw holes of the bone plate under the skin, reduce the size of a surgical incision, and shorten an operation time, so as to effectively help a clinician to perform an endosseous fixation surgery, reduce complications, improve the operation efficiency, and decrease the waste of medical resources.

The bone fixation guide device of the present invention can be used to perform a minimally invasive surgery for fracture fixation, and can place the bone plate at a subcutaneous focus by combining the handheld holder, the first guide sleeve, and the bone plate. After the handheld portion, the first guide sleeve, and the bone plate are combined, the positions of openings of the guide plate correspond to the positions of screw holes of the bone plate under the skin. The guide probe is then used to determine an approximate position of a following bone screw on the skin, and the second guide sleeve first passes through the skin and then is combined with the bone plate, so as to perform drilling and to accurately lock the bone screws into a bone body vertically. Therefore, the design of the bone fixation guide device has the following advantages: 1. The bone fixation guide device has a simple structure, is easy to be operated, and can improve the operation efficiency. 2. The bone fixation guide device can be used with surgical instruments, so as to accurately obtain the screw holes of the bone plate under the skin and perform drilling immediately without having to remove the bone fixation guide device after surgical alignment, and thus to improve surgical convenience. 3. The size of the incision is reduced, and the time that a patient is exposed to an X-ray and the operation time are shortened.

The present invention provides a bone fixation guide device for a minimally invasive treatment of a bone fracture, thereby can accurately positioning the screw holes of the bone plate under the skin. By taking the fixation surgery of fracture at the end of radius as an example, the bone fixation guide device can reduce the size of a surgical incision to be less than 50%, and shorten an operation time from 1.5 hours to 0.5 hours, so as to effectively help a clinician to perform an endosseous fixation surgery, reduce complications, improve the operation efficiency, and decrease the waste of medical resources.

The above merely describes embodiments of technical measures employed by the present invention for solving the problem, but is not to limit the scope of the implementation of the present invention. Equivalent variations and modifications consistent with the literal meaning of the claims of the present invention or made according to the scope of the present invention should fall within the scope of the present invention.

What is claimed is:

1. A bone fixation guide device adapted for fixing a bone plate on a bone, the bone plate having at least two screw holes, the bone fixation guide device comprising:
a handheld holder having a through hole;
a first guide sleeve having a guide hole, adapted for passing through the through hole of the handheld holder, and fixed to the bone plate; and
a guide plate having a fixing portion and at least two openings, the fixing portion adapted for being assembled on first guide sleeve, whereby the guide plate is disposed on the handheld holder, wherein the screw holes of the bone plate correspond to the openings of the guide plate.

2. The bone fixation guide device according to claim 1, further comprising:
a guide probe adapted for passing through the guide hole of the first guide sleeve, and temporarily fixing the bone plate on the bone;
a marker adapted for passing through one of the openings, and making a mark on a skin; and
a second guide sleeve having a guide hole, and adapted for being fixed to the bone plate, so that the guide hole of the second guide sleeve is located above one of the screw holes of the bone plate, and the one of the screw holes corresponds to the one of the openings.

3. The bone fixation guide device according to claim 1, wherein an external thread of the first guide sleeve is screwed and fixed to an internal thread of the bone plate.

4. The bone fixation guide device according to claim 3, wherein a positioning protrusion of the bone plate is stuck in a positioning recess of the handheld holder, or a positioning protrusion of the handheld holder is stuck in a positioning recess of the bone plate.

5. The bone fixation guide device according to claim 4, wherein a positioning column of the guide plate is stuck in a positioning hole of the handheld holder or a positioning column of the handheld holder is stuck in a positioning hole of the guide plate, so as to ensure that the guide plate is parallel to the bone plate.

6. The bone fixation guide device according to claim 1, wherein a grip is provided at a side surface of the handheld holder.

7. The bone fixation guide device according to claim 1, further comprising:
a sliding sleeve having a through hole, and adapted for contacting with the bone plate, so that the through hole of the sliding sleeve is located above the screw holes of the bone plate; and
a sliding rod adapted for passing through the through hole of the sliding sleeve, and being inserted into the screw hole of the bone plate.

8. The bone fixation guide device according to claim 2, further comprising:
a sliding sleeve having a through hole, and adapted for contacting with the bone plate, so that the through hole of the sliding sleeve is located above the screw holes of the bone plate; and
a sliding rod adapted for passing through the through hole of the sliding sleeve, and being inserted into the screw hole of the bone plate.

9. A bone fixation guide device for fixing a bone plate on a bone, the bone plate having two screw holes, the bone fixation guide device comprising:
a handheld holder having a through hole;
a first guide sleeve having a guide hole, wherein an end of the first guide sleeve passes through the through hole of the handheld holder to contact with one of the screw holes of the bone plate;
a guide plate and having a fixing portion and two openings, the fixing portion adapted for being assembled on first guide sleeve, so that the guide plate is disposed above the bone plate, and the two openings of the guide plate are corresponding to the two screw holes of the bone plate;
a guide probe adapted for passing through the guide hole of the first guide sleeve; and
a second guide sleeve having a guide hole, wherein an end of the second guide sleeve adapted to contact with the other screw hole of the bone plate.

10. The bone fixation guide device according to claim 9, wherein an external thread of the first guide sleeve is screwed and fixed to an internal thread of the bone plate.

11. The bone fixation guide device according to claim 9, wherein a positioning protrusion of the bone plate is stuck in a positioning recess of the handheld holder, or a positioning protrusion of the handheld holder is stuck in a positioning recess of the bone plate.
